(19) United States
(12) Patent Application Publication
(10) Pub. No.: US 2010/0130967 A1
(43) Pub. Date: May 27, 2010

(54) Method and Apparatus for Enhanced Corneal Accommodation

(76) Inventors: Mathias Glasmacher, Reilingen (DE); Frieder Loesel, Mannheim (DE)

Correspondence Address: NYDEGGER & ASSOCIATES, 348 OLIVE STREET, SAN DIEGO, CA, 92103, US

(21) Appl. No.: 12/275,997

(22) Filed: Nov. 21, 2008

(51) Int. Cl. A61F 9/01; A61B 18/20

(52) U.S. Cl. 606/5

(57) ABSTRACT

A system and method for improving the accommodative power of a focusing unit (e.g. an eye) involves increasing the flexibility of a first optical element in the unit (e.g. the cornea of the eye). Specifically, the needed flexibility is determined from diagnostic data, and the first optical element is structurally weakened according to the data. With this weakened structure (i.e. increased flexibility), the first element is better able to comply with configuration changes in a second optical element in the focusing unit (e.g. the lens of the eye). The consequence is, improved accommodation. For the present invention, the improved compliance to achieve optimal accommodation is accomplished either by performing appropriate LIOB on stromal tissue in the eye, or by application of a topical medium to selected areas on the anterior surface of the eye.

### FIELD OF THE INVENTION

The present invention pertains generally to systems and methods for improving the visual performance of an optical unit. More particularly, the present invention pertains to systems and methods that facilitate the accommodation of a human eye. The present invention is particularly, but not exclusively, useful for systems and methods that alter physical characteristics of the cornea of a human eye, to facilitate accommodation by the lens of the eye.

### BACKGROUND OF THE INVENTION

Ocular accommodation refers to the ability of an eye to change its effective focal length, and to thereby see objects distinctly at varying distances. As is well known, the predominant anatomical mechanism for achieving accommodation involves changes in the configuration of the crystalline lens in the eye. Specifically, this configuration change results from a contraction of the ciliary muscle that causes the curvature of the lens surfaces to increase. There is, however, evidence that additional factors may be involved in accommodation. A recent article by Akihiro Yasuda, Tatsuo Yamaguchi, and Kishiko Ohkoshi, entitled “Changes in corneal curvature in accommodation” suggests that changes in corneal curvature in accommodation, participate in the mechanism of accommodation (see J Cataract Refract © 2003 ASCRS and ESCRS). Accordingly, current thinking is that the physical configurations of both the cornea and the lens of an eye work together with each other for accommodation.

With increased age, the crystalline lens of an eye becomes more rigid, and its accommodative amplitude declines. Consequently, the configuration compliance of the cornea with the lens that is required for effective accommodation may be adversely affected. If so, a higher flexibility in the structure of the cornea is needed to facilitate the necessary compliance.

It is well known that cornea flexibility can be improved by selectively weakening the cornea in accordance with appropriate diagnostic data. For example, U.S. patent application Ser. No. 12/127,539, which was filed on May 7, 2008 by Bille et al. for an invention entitled “System and Method for Reshaping a Cornea Using a Combination of LIOB and Structural Change Procedures,” and which is assigned to the same assignee as the present invention, discloses weakening of the cornea for improved flexibility. Further, using diagnostic data, it is also well known that mathematical models can be useful for predicting configuration changes of anatomical structures in dynamic situations. For example, U.S. patent application Ser. No. 12/143,600, which was filed on Jun. 20, 2008 by Bille et al. for an invention entitled “Generalized Modeling of the Cornea,” and which is assigned to the same assignee as the present invention, discloses such a use for a predictive mathematical model.

In light of the above, it is an object of the present invention to provide a system and method to facilitate compliance between two different elements of an optical unit during changes in the unit's focal distance. Another object of the present invention is to alter the physical characteristics of a first optical element (e.g. the cornea of an eye) to facilitate its compliance with changes in the physical characteristics of a second optical element (e.g. the lens of the eye), for improved optical performance of the second element (e.g. the lens). Yet another object of the present invention is to provide a patient with a stronger change of focal power during the accommodation process for improved near vision (i.e. improved reading ability). Still another object of the present invention is to provide a system, and its method of use, for improving the accommodation performance of an eye that is relatively easy to implement, is simple to use, and is comparatively cost effective.

### SUMMARY OF THE INVENTION

In accordance with the present invention, a system and method for improving the ocular accommodation of an eye involves facilitating compliance of a first optical element (e.g. the cornea of the eye) with a second optical element (e.g. the lens of the eye). In particular, facilitating this compliance is a consequence of improving the flexibility of the cornea (e.g. first optical element). And, more specifically, it is accomplished by minimizing optical interference of the cornea with the lens. Structurally, the necessary compliance is achieved by weakening a selected portion of the cornea (i.e. first optical element) to achieve optimal accommodation.

As a first step in the method of the present invention, at least one physical characteristic of the cornea (first optical element) is measured. For the present invention the physical characteristic is envisioned to be selected from a group consisting of the refraction of an eye, the topography of an eye, the corneal thickness profile of an eye, the wavefront aberrations of an eye, and the biomechanical properties of an eye. Preferably, measurements of the physical characteristic are made on the cornea while the lens transitions through an accommodation range of approximately fifteen diopters. The diagnostic data that is collected during these measurements can then be used as input to a Finite Element Model (FEM). Output from the FEM will be geometric parameters for the cornea that, in turn, can be used to identify required alterations of the cornea to facilitate its compliance with the lens during accommodation.

After the diagnostic data has been collected and the geometric parameters have been identified, the cornea can be appropriately altered (i.e. weakened). To do this, in one embodiment of the present invention, a laser unit is used to perform Laser Induced Optical Breakdown (LIOB) in stromal tissue of the cornea. Specifically, this LIOB is accomplished over at least one defined intrastromal surface, such as ring cuts, radial cuts, ring section segments, horizontal layers or combinations thereof. In another embodiment of the present invention, the cornea is weakened by applying a topical medium to selected areas of its anterior surface. In both embodiments, the purpose is to weaken the cornea so it will be more compliant with changes in lens configuration and, thus, improve accommodation of the eye.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a schematic presentation of a system in accordance with the present invention shown operationally positioned for interaction with an eye (shown in cross section);

FIG. 2A is a top plan view of the anterior surface of an eye as seen along the line 2-2 in FIG. 1 with representative cylindrical intrastromal LIOB cuts shown end-on;

FIG. 2B is a view as seen in FIG. 2A showing representative ring segment LIOB cuts;

FIG. 2C is a view as seen in FIG. 2A showing representative radial LIOB cuts; and

FIG. 3 is a cross section view of the cornea of an eye as seen along the line 3-3 in FIG. 1 showing horizontal layer LIOB cuts in the stroma of an eye.

### DESCRIPTION OF THE PREFERRED EMBODIMENTS

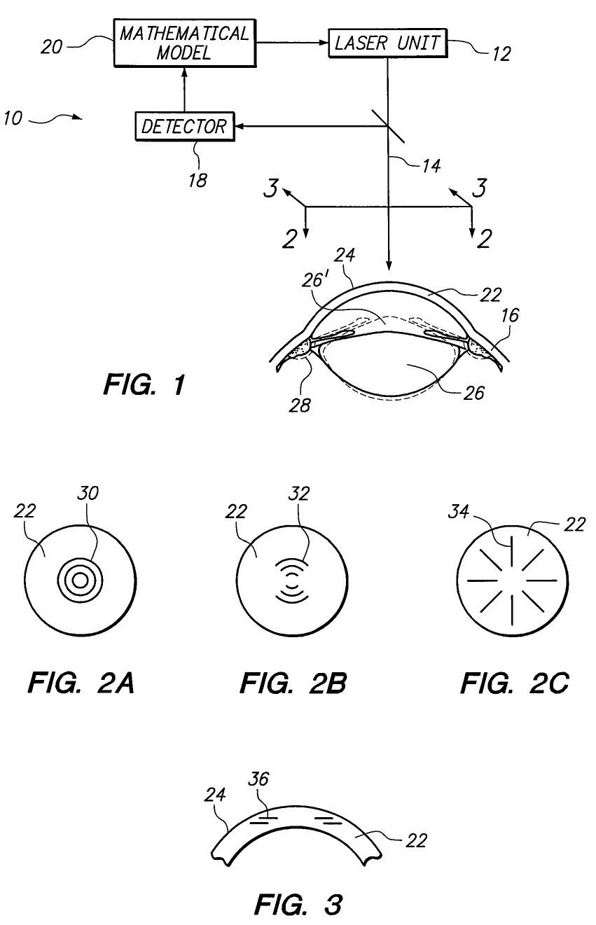

Referring initially to FIG. 1 a system for improving the accommodative power of a focusing unit is shown and is generally designated 10. As shown, the system 10 includes a laser unit 12 that is positioned to direct a light beam along a beam path 14 toward an eye 16. Further, FIG. 1 shows that light reflected from the eye 16 can be directed toward a detector 18 for a diagnostic analysis of the eye 16. The diagnostic data that is collected and analyzed by the detector 18 can then be used as input to a mathematical model 20.

In accordance with the present invention, the laser unit 12 can be considered as performing two different functions. For one, the unit 12 can function as a light source that is used for generating the diagnostic data. In this capacity, the unit 12 can be any of several type light sources well known in the pertinent art. For another function, the laser unit 12 can function as a surgical laser that can be used to alter stromal tissue within the cornea 22 of the eye 16. In this latter capacity, the laser unit 12 preferably generates a pulsed laser beam having ultra-short pulses (e.g. femto-second duration pulses). As envisioned for the system 10, the alteration of stromal tissue will be accomplished by a well known phenomenon generally referred to as Laser Induced Optical Breakdown (LIOB).

For the system 10 of the present invention, the diagnostic data that is collected and analyzed by the detector 18 is envisioned to include various anatomical aspects of the cornea 22 of the eye 16. For example, using wavefront analysis, this data can include refractive properties of the eye 16, and of the cornea 22. Also, the data can include the topography of the anterior surface 24 of the cornea 22, the corneal thickness profile (pachymetry), and wavefront aberrations as well as various biomechanical properties of the eye 16. Preferably, this data is collected while the eye 16 is caused to experience accommodation through an accommodation range that may be as much as fifteen diopters. In any event, the collected diagnostic data can then be used to determine how the cornea 22 should be altered (i.e. weakened) for purposes of the present invention. More specifically, using the diagnostic data, geometric parameters for structurally altering the cornea 22 are obtained. Preferably, this is done using a mathematical, predictive model such as the finite element model disclosed and claimed in U.S. patent application Ser. No. 12/143,600, and referred to above.

For one embodiment of the present invention, the geometric parameters are used to establish patterns of Laser Induced Optical Breakdown (LIOB) in stromal tissue of the cornea 22. Recall, these parameters are based on diagnostic data and are preferably obtained from the predictive model 20. In another embodiment, these same geometric parameters can be used to identify selected areas of the anterior surface 24 of the cornea 22 where a topical agent (e.g. an enzyme) can be applied to weaken the cornea 22, to thereby improve its flexibility. For both embodiments, the object is to weaken corneal tissue for increased flexibility of the cornea 22. As intended for the present invention, this improved flexibility facilitates accommodation of the eye 16.

Still referring to FIG. 1, it will be appreciated that an accommodation mechanism for the eye 16 changes the anatomical configuration of the lens 26 of the eye 16. As is well known, this configuration change is caused by contractions of the ciliary body (muscle) 28. The result is a configuration change for the lens 26 that is represented in FIG. 1 by variations between the configuration of lens 26 (solid line) and the configuration of lens 26′ (dashed line). As mentioned above, an accommodation of the eye 16 will typically be within an accommodation range of approximately fifteen diopters.

As envisioned for the present invention, accommodation provided by configuration changes of the lens 26 will be facilitated when the cornea 22 complies with these changes. This requires flexibility on the part of the cornea 22. And, according to the present invention, the necessary flexibility is achieved by weakening selected portions of the cornea 22. The exact locations for weakening the cornea 22, and the extent or scope of such weakening will be determined by an analysis of the diagnostic data. Several examples of possible alterations for weakening the cornea 22 are illustrated in FIGS. 2A-C and FIG. 3. Respectively, these possible alterations include LIOB for rings and cylinders 30 (FIG. 2A), ring section segments 32 (FIG. 2B), radial cuts 34 (FIG. 2C), and horizontal layers 36 (FIG. 3).

While the particular Method and Apparatus for Enhanced Corneal Accommodation as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A method for facilitating simultaneous, configuration changes in first and second optical elements of a focusing unit to achieve an optimal accommodation for the focusing unit, the method comprising the steps of:
measuring at least one physical characteristic of the first optical element to obtain diagnostic data; and
weakening a structural aspect of the first optical element, wherein the extent and scope of the weakening is based on the diagnostic data from the measuring step, and is accomplished to facilitate a compliance in the configuration of the first optical element with the configuration of the second optical element to achieve an optimal accommodation for the focusing unit.

2. A method as recited in claim 1 wherein the first optical element has an exposed anterior surface and the weakening step is accomplished by applying a topical medium to selected areas of the anterior surface.

3. A method as recited in claim 1 wherein the first optical element has an anterior surface with a posterior surface substantially parallel thereto, and wherein the weakening step is accomplished by selectively altering material located between the anterior surface and the posterior surface of the first optical element.

4. A method as recited in claim 3 wherein altering of the material is accomplished by a Laser Induced Optical Breakdown (LIOB) of the material.

5. A method as recited in claim 4 wherein LIOB is accomplished in the material over at least one defined surface, wherein the surface is selected from a group consisting of ring cuts, radial cuts, ring section segments, horizontal layers and combinations thereof.

6. A method as recited in claim 1 wherein the physical characteristic is selected from a group consisting of the refraction of an eye, the topography of an eye, the corneal thickness profile of an eye, the wavefront aberrations of an eye, and the biomechanical properties of an eye.

7. A method as recited in claim 1 wherein the second optical element is a lens having a curved anterior surface, and the method further comprises the step of measuring the curvature of the anterior surface for inclusion in the data.

8. A method as recited in claim 1 wherein the first optical element is a cornea of an eye and the second optical element is a lens of the eye.

9. A method as recited in claim 1 wherein the measuring step is accomplished during a transition of the focusing unit through an accommodation range.

10. A method as recited in claim 9 wherein the accommodation range is approximately fifteen diopters.

11. A method for facilitating compliance of a first optical element with a second optical element in response to a configuration change of the second optical element during a focusing accommodation which comprises the steps of:
measuring at least one physical characteristic of the first optical element to obtain diagnostic data;
inputting the data from the measuring step into a mathematical model to obtain geometric parameters for the first optical element; and
altering the first optical element in accordance with the geometric parameters obtained during the inputting step to facilitate compliance of the first optical element with the second optical element within a predetermined accommodation range.

12. A method as recited in claim 11 wherein the first optical element is a cornea of an eye and the altering step is accomplished using a laser unit to perform Laser Induced Optical Breakdown (LIOB) in stromal tissue of the cornea.

13. A method as recited in claim 12 wherein the physical characteristic is selected from a group consisting of the refraction of the eye, the topography of the eye, the corneal thickness profile of the eye, the wavefront aberrations of the eye, and the biomechanical properties of the eye.

14. A method as recited in claim 12 wherein the cornea has an anterior surface with a posterior surface substantially parallel thereto, and wherein the altering step is selectively accomplished by LIOB of material located between the anterior surface and the posterior surface of the first optical element, and further wherein LIOB is accomplished in the material over at least one defined surface, wherein the surface is selected from a group consisting of ring cuts, radial cuts, ring section segments, horizontal layers and combinations thereof.

15. A method as recited in claim 11 wherein the mathematical model is a finite element model.

16. A method as recited in claim 11 wherein the first optical element has an exposed anterior surface and the altering step is accomplished by applying a topical medium to selected areas of the anterior surface.

17. A method as recited in claim 11 wherein the measuring step is accomplished during a transition of the second optical element through an accommodation range of approximately fifteen diopters.

18. A system for facilitating compliance of a cornea of an eye with a lens of the eye in response to a configuration change of the lens during a focusing accommodation which comprises:
a means for measuring at least one physical characteristic of the cornea to obtain diagnostic data;
a mathematical model for receiving the diagnostic data to generate geometric parameters for the cornea; and
a laser unit for altering stromal tissue in the cornea with Laser Induced Optical Breakdown (LIOB) in accordance with the geometric parameters generated by the model, to facilitate compliance of the cornea with the lens within a predetermined accommodation range.

19. A system as recited in claim 18 wherein LIOB is accomplished in the material over at least one defined surface, wherein the surface is selected from a group consisting of ring cuts, radial cuts, ring section segments, horizontal layers and combinations thereof.

20. A system as recited in claim 18 wherein the physical characteristic is measured during a transition of the lens through an accommodation range of approximately fifteen diopters.
